(19) United States
(12) Patent Application Publication
(10) Pub. No.: US 2023/0137888 A1
(43) Pub. Date: May 4, 2023

(54) COMPOSITION FOR UPPER RESPIRATORY TRACT ADMINISTRATION AND METHOD THEREOF

(71) Applicant: ORIGINAL BIOMEDICALS CO., LTD., Tainan City (TW)

(72) Inventors: Jia-Long Chen, Tainan City (TW); Wei-Chuan Liao, Tainan City (TW); Tzu-Hui Sun, Tainan City (TW); Chia-Hung Chen, Tainan City (TW); Chau-Hui Wang, Tainan City (TW); Hsiao-Pao Yen, Tainan City (TW)

(21) Appl. No.: 17/972,733

(22) Filed: Oct. 25, 2022

Related U.S. Application Data
(60) Provisional application No. 63/273,317, filed on Oct. 29, 2021.

(51) Int. Cl. A61K 9/00; A61K 31/714; A61K 31/16; A61P 11/00

(52) U.S. Cl. CPC A61K 9/0078; A61K 31/16; A61K 31/714; A61P 11/00

(57) ABSTRACT

The present invention discloses a composition for upper respiratory tract administration comprising a cyanide antidote and a metal chelator, and the composition is used for cure or protection of fire injury. When carried by a proper carrier, the composition is administrated to a subject in need via forms including a spray, an inhaler, or drops so as to prevent or cure injuries caused by hazardous substances in a fireground.

### CROSS REFERENCE TO RELATED APPLICATION

This application claims the benefit of Provisional Application Ser. No. 63/273,317, filed Oct. 29, 2021.

### FIELD OF THE INVENTION

The present invention relates to a composition, especially relating to a composition for upper respiratory tract administration which is applied to fire injury cure or protection.

### BACKGROUND OF THE INVENTION

In a fireground, heavy smoke deriving from completely burnt and incompletely burnt items contains solid smoke particles, toxic gases and a large amount of smog generated by splitting decomposition at high temperature. The heavy smoke can severely damage health of firefighters, on-site victims or residents in the neighborhood.

In recent years, species and amounts of toxic gases in a fireground have been dramatically increased because synthetic building materials and polymer materials are widely used in house furnishing, offices and public places of entertainment. For example, the toxic gases include acrylicin, acrylonitrile, benzene, formaldehyde, sulfur dioxide, hydrogen cyanide, dioxin, polycyclic aromatic hydrocarbons, etc.. These toxic gases can directly or indirectly cause irreversible damages such as illness, degeneration, or chronic disease in a personnel. Amongst these toxic gases, hydrogen cyanide (HCN) is an instant toxic substance. HCN demonstrates high affinity to ferric ions in cytochrome oxidases. When HCN enters a human body, it forms stable complex with cytochrome oxidases and interferes with oxygen binding cytochrome oxidases, which eventually results in inaccessibility of oxygen to the body cells.

Kelocyanor® having dicobalt edetate is an injective form cyanide antidote. Cobalt ions form stable complex with cyanide in the blood, and the complex is excreted out of the body via urine. However, cobalt exists in an ionic form remains unpredictably toxic to an individual.

Disclosed in Pat. No. GB 1404324 is a process for the diagnosis of chronic hypercyanogenesis. A composition having sodium thiosulphate and hydroxocobalamine is used to track the variating trend of CN⁻ concentration in the blood. The composition is administered at a single dose or multiple doses via intramuscular injection. Nonetheless, hypercyanogenesis deteriorates after multiple-dose administration, which implicates its inapplicability to chronic therapy.

Disclosed in Pat. No. US5834448 is a new dosage form of hydroxocobalamin and its antidotal use in cyanide poisoning. The hydroxocobalamin is freeze-dried in an acidic medium so that it can be instantly redissolved in a neutral saline solution. Stability of the hydroxocobalamin in preservation is significantly improved. However, the hydroxocobalamin-based pharmaceutical compositions provided therein is administered via intravenous injection. The dosage form limits its applicability in an environment where cyanide exposure reaches hazardous level. The hydroxocobalamin-based pharmaceutical compositions can not be administered directly into the environment or to subjects in need by inhalation to achieve protective or therapeutic effect.

In addition to toxic gases as previously described, heavy smoke in a fireground further include particulate matters, dust, fibers, glass fibers or other hazardous substances. These hazardous substances is not only life-threatening to on-site disaster relief workers, but also detrimental to post-disaster inspectors. During post-disaster inspection, atmosphere at fireground would be full of smoke substances such as dust, particulate matters or fibers when fire debris is moved, which may harm post-disaster inspectors’ health when they are exposed to or even inhale those hazardous substances.

According to “Standard Operating Procedures for Fire Investigation and Identification” issued by the Fire Department of the Ministry of the Interior, R.O.C., inspectors must be equipped with antigas cartridges when entering a fireground for post-disaster investigation. The antigas cartridge can efficiently filter most of toxic gases such as organic gases, chlorine, hydrochloric acid, sulfur dioxide, hydrogen sulfide, or chlorine dioxide. Nevertheless, in practical use, it requires a long time for antigas cartridge to filter toxic gases such as hydrogen chloride, hydrogen cyanide and acrolein, which limits its protective effect and also increases risk of inhaling toxic gases by personnel.

On top of using antigas cartridge, current means of protecting personnel in exposure to hazardous substances in a fireground further includes physical isolation such as wearing respiratory system protective equipment. However, heavy smoke in a fireground is a mixture of particulate matters and toxic gases, and the averaged particle size is less than 10 um. Hence, current physical protective equipment remains unable to completely filter particulate matters in fireground atmosphere. Meanwhile, solid microparticles in the heavy smoke are irritative to upper respiratory tract and cause an abundance of mucous secretion which may congest the upper respiratory tract.

Withal, in a fireground, inhalation of hydrogen cyanide and free radicals from splitting decomposition is also considered a main cause of instantaneous injury in that free radicals leads to strong oxidative stress to human body tissues. When abundance of internal free radicals exceeds a normal range, “free radical chain reaction” starts to oxidize proteins, carbohydrates or lipids and new free radicals are generated thereby. Excessive free radicals bring damage to genetic substances (such as DNA), lipid peroxidation, enzyme deactivation, and inflammation due to abnormal activity of immune cells including monocytes or macrophages. “Free radical chain reaction” jeopardizes an individual’s health from both a short-term and a long-term perspective.

With regard to this, to overcome the dilemma that current technology can not guarantee protection against both solid and gaseous hazardous substances in fireground smoke, development of a novel means to eliminate and to neutralize both kinds of hazardous substances is urgently required. The novel means does not only protect firefighters’ health and prevent them from heavy smoke damage, but also reverse damage resulting from inhalation of the heavy smoke, and injuries are effectively cured thereupon.

### SUMMARY OF THE INVENTION

Considering that heavy smoke in a fireground is a complicated damage form to on-site personnel’s life and safety, the instant inventor manages to develop a new type of means for comprehensive protection and treatment in addition to current means of hydrogen cyanide elimination. The present invention aims to provide a means for elimination and neutralization of both solid and gaseous hazardous substances in heavy smoke. In view of this, the present invention provides a composition for upper respiratory tract administration comprising a cyanide antidote and a metal chelator, and the composition is used for cure or protection from fire injury.

In various embodiments, the cyanide antidote is selected from a group consisting of hydroxocobalamin, dicobalt edetate, cobinamide, aquohydroxocobinamide, dinitrocobinamide, methemoglobin, sodium nitrite, amyl nitrite, dimethyl aminophenol, sodium thiosulfate and glutathione.

In various embodiments, the metal ion chelator is selected from a group consisting of deferoxamine, deferiprone and deferasirox.

In some embodiments, the composition further comprises an expectorant selected from a group consisting of KI, iodinated glycerol, glyceryl guaiacolate, guaifenesin, ambroxol, bromhexine, N-acetylcysteine and lysozyme.

In some embodiments, the composition further comprises a carrier, wherein the carrier comprising ionized water, secondary water, ultra-pure water or buffer solution. Preferably, the buffer solution having a buffer solute selected from a group consisting of phosphate, tris(hydroxymethyl)aminopropanesulfonic acid, dihydroxyethylglycine, tris(hydroxymethyl)aminomethane, tris(hydroxymethyl)methylglycine, 4-hydroxyethylpiperazineethanesulfonic acid acid, n-tris(hydroxymethyl)methyl-2-aminoethanesulfonic acid, 3-(n-morpholine)ethanesulfonic acid, piperazine-n,n′-bis(2-ethanesulfonic acid), dimethylarsinic acid, sodium citrate and 2-morpholineethanesulfonic acid

In various embodiments, the composition is further used for protection and cure confronting with potential exposure to an environment of high concentration cyanide.

Additional embodiments of the present invention disclose a method for upper respiratory tract administration comprising administrating the aforementioned composition to a subject in need at a time point until the composition reaches an effective dose so as to suppress injury upon the subject in need in exposure to toxic gases, wherein the time point is prior to or post exposing the subject in need to the toxic gases.

In various embodiments, the toxic gases comprising particulate matters, free radicals or hydrogen cyanide.

Preferably, the route of administration comprises biological fluid absorption or mucosal absorption.

Preferably, the subject in need comprises a mammal.

Current means to eliminate hydrogen cyanide relies on solution injection as a main delivery form, such as intramuscular injection or intravenous injection. It requires time for effects to emerge, and instability of the dosage forms is also concerned, which insuperably obstructs user end as well as producer end. Besides, cyanide poisoning is not the only risk in a fireground, other harmful factors including particulate matters and free radicals are also difficult for prior technologies to overcome.

Places having high tendency of cyanide poisoning includes workplaces of particular occupations, such as electroplating, metallurgy, plastic industry. Besides acute toxication, long-term exposure of a personnel in an environment of potential cyanide poisoning is also taken into considerations. Wherefore, drug administration regards not only to treatment in acute poisoning phase, but also to precaution of poisoning prior to personnel’s entry into these workplaces. Prior technologies with injection dosage forms remain ineffectual in the above situations.

The composition for upper respiratory tract administration and the method thereof in the present invention provides solutions for curing and prevention of acute or chronic cyanide poisoning, addressing issues of inexpedient cyanide poisoning treatment encountered by prior arts. Additionally, metal ion chelator blocks free radical chain reaction, and relieves oxidative stress of tissues and cells in a human body. Expectorant promotes mucosal excretion to eliminate particulate matters’ irritation and jamming in upper respiratory tract so that the upper respiratory tract maintains unobstructed.

In sum, the composition for upper respiratory tract administration and the method thereof in the present invention overcome limitations of dosage forms in prior technologies. The composition rapidly enters a body at a low dose and achieves protective and therapeutic effect in a normal situation, fireground or environment of trace cyanides.

Furthermore, the composition for upper respiratory tract administration is not limited to on-site environment or subject’s status during administration. The composition is nebulized for an subject to inhale when necessary. The composition is absorbed via subject’s lung and enters the blood circulation, or forms a protective layer on the surface of upper respiratory tract and alveoli. Both fire injury protection and treatment can be achieved, and first aid of acute poisoning as well as prevention in long-term exposure are both taken into account.

### BRIEF DESCRIPTIONS OF DRAWINGS

FIG. 1A illustrates an exemplary environment of gaseous cyanide in a barrel. FIG. 1B illustrates an exemplary administration of the composition for upper respiratory tract in a nebulized form.

### DETAILED DESCRIPTION OF THE INVENTION

The present disclosure provides a composition for upper respiratory tract administration comprising a cyanide antidote and a metal chelator, and the composition is used for cure or protection from fire injury; preferably, the composition comprises 5 to 10 parts by weight cyanide antidote, and 1 to 5 parts by weight metal ion chelator.

In various embodiments, the cyanide antidote is selected from a group consisting of hydroxocobalamin, dicobalt edetate, cobinamide, aquohydroxocobinamide, dinitrocobinamide, methemoglobin, sodium nitrite, amyl nitrite, dimethyl aminophenol, sodium thiosulfate and glutathione. Preferably, the cyanide antidote comprises hydroxocobalamin. Concretely, the cyanide antidote forms complexes with cyanide ion via ligand binding so as to neutralize toxicity of cyanide ions. Competition of the cyanide ions with oxygen for divalent ferric ions (Fe²⁺) is avoided thereby. For example, hydroxocobalamin, a.k.a. vitamin B 12a, has a cobalt ion at its chemical structural center. The cobalt ion binds to cyanide ions (CN⁻) and cyanocobalamin, a.k.a. vitamin B12, is formed. Cyanocobalamin is detoxified and excreted from the body along with water. Meanwhile, hydroxocobalamin rapidly enters mitochondria and binds to cyanides so that cellular oxidative metabolism is restored.

In various embodiments, the metal ion chelator is selected from a group consisting of deferoxamine, deferiprone and deferasirox. Preferably, the metal ion chelator comprises deferoxamine. Specifically, a large amount of free radicals are generated by splitting decomposition at high temperature in a fireground. Fenton’s reaction initiates after the free radicals are inhaled into the body and react with internal metal ions, such as ferric ions or copper ions. Fenton’s reaction produces more free radicals and causes more oxidative stress over tissues and cells, which eventually leads to free radical damage. To obviate free radical damages, the metal ion chelator in the composition, such as deferoxamine, competes with free radicals for ferric ions in blood stream. Thus, the free radical chain reaction is blocked and cells are protected from oxidative stress.

In additional embodiments, to remove excessive mucous secreted by the irritated upper respiratory tract when a subject inhales hazardous substances such as particulate matters, the composition further comprises an expectorant selected from a group consisting of KI, iodinated glycerol, glyceryl guaiacolate, guaifenesin, ambroxol, bromhexine, N-acetylcysteine and lysozyme. Preferably, the expectorant comprises N-acetylcysteine. More preferably, the composition comprises 5 to 10 parts by weight cyanide antidote, 1 to 5 parts by weight metal ion chelator and 5 to 10 parts by weight expectorant.

Particularly speaking, an expectorant breaks disulfide bonds to lower mucous viscosity so that excretion of sputum is promoted. For instance, N-acetylcysteine has a thiol group (—SH) whose sulfur atom possesses a larger 3 s/3 p hybridized orbital, and hydrogen atom has a smaller 1 s orbital. Therefore, S—H bond is weak and tends to be oxidized, and the lone pair on sulfur atom is unmasked to reduce and break disulfide bond, which reduces viscosity of sputum. Bromhexine stimulates bronchial mucous secretion and further dissolves the mucous so as to diminish mucous viscosity. Also, pseudostratified columnar epithelium is activated by bromhexine to drive the mucous out of the body. Regarding to ambroxol, as an active metabolite of bromhexine, ambroxol presents similar characteristics of mucous elimination and secretion dissolution. Ambroxol is able to accelerate mucous excretion out of respiratory tract and minimize the amount of remaining mucous. Amborxol enhances removal of sputum so that respiration is improved. Likewise, lysozyme is a native antibacterial enzyme existing in human secretion such as tears or saliva. Carbon-oxygen bond of the substrate is broken by side chains of Glu35 and Asp52 in lysozyme protein sequence, and mucous is then decomposed so that excretion of sputum is promoted. In addition to mucous decomposition, other expectorants achieves at rapid sputum excretion through stimulating cells on respiratory tract surface, such as respiratory epithelium cells. For example, glyceryl guaiacolate directly excites bronchial secretory cells (e.g. Clara cell) and the subject’s reflex to dilute sputum.

In the present disclosure, the composition further comprises a carrier so as to transport the composition into a human body in a variety of dosage forms. The dosage form can be a spray, an inhaler, a nebulizer, or drops, and not limited to this. Preferably, the inhaler is a metered-dose inhaler, a spray inhaler or a dry powder inhaler. The carrier may be a solvent or solution with appropriate solubility for the active ingredient, but not limited to this. In various embodiments, the carrier comprises ionized water, secondary water, ultra-pure water or buffer solution. Preferably, the buffer solution has a buffer solute selected from a group consisting of phosphate, tris(hydroxymethyl)aminopropanesulfonic acid, dihydroxyethylglycine, tris(hydroxymethyl)aminomethane, tris(hydroxymethyl)methylglycine, 4-hydroxyethylpiperazineethanesulfonic acid acid, n-tris(hydroxymethyl)methyl-2-aminoethanesulfonic acid, 3-(n-morpholine)ethanesulfonic acid, piperazine-n,n′-bis(2-ethanesulfonic acid), dimethylarsinic acid, sodium citrate and 2-morpholineethanesulfonic acid

In various embodiments, the composition comprises 5 to 10 parts by weight the cyanide antidote, 1 to 5 parts by weight the metal ion chelator, 5 to 10 parts by weight the expectorant and 60 to 200 parts by weight the carrier.

Similarly, volatility of cyanides is harmful to on-site personnel in various occupational workplaces. Taking hydrogen cyanide for example, it tends to evaporate and dissolve in water at temperature above 28° C. Hydrogen cyanide can be released by cyanogenetic glucoside. Cyanogenetic glucoside is a natural ingredient in some economical plants, such as bitter almonds, sorghum, cassava, Lima beans, drupe or bamboo shoots. For instance, root of cassava contains aminolevulinic acid dehydratase (ALAD) which enzymatically digests cyanogenetic glucoside and releases cyanoalcohol. Cyanoalcohol breaks up into hydrogen cyanide in low acidic environment, which eventually increases concentration of cyanides in atmosphere in a cassava-processing workplace. In view of this, the composition for upper respiratory tract administration in the present disclosure offers a solution for protection and treatment to personnel in the aforementioned workplaces where the personnel is potentially exposed to high concentration of cyanides, and minimizes occupational injury thereof. The workplaces can be exemplified by places for cassava processing, sorghum processing, or drupe processing, or further by places for photography, electroplating, hydrogen sulfide manufacturing, fumigant manufacturing, pharmaceutical manufacturing, papermaking, dyeing, rodenticide operation, insecticide operation, blast furnace gas operation, cyanide manufacturing, nitric acid manufacturing, rubber plastic manufacturing, synthetic fiber manufacturing, leather manufacturing, pesticide industry, gold and silver processing, metal surface hardening, Prussian blue fiber printing, metallurgical operation, bone phosphoric acid extraction, organic nitrogen compound manufacturing, soda manufacturing, gold inlay operation, biochemical weapons manufacturing, lighting gas manufacturing, or nitrocellulose burning operations, but not limited to this.

In order to increase solubility of the carrier, the composition further comprises an acid-base adjuster so as to maintain the composition at weak basic state. Preferably, the composition can be adjusted to pH 7.0 to 9.0, and more preferably to pH 7.5 to 8.5. The acid-base adjuster can be exemplified by citric acid, acetic acid, phosphoric acid, carbonic acid, hydrochloric acid, tartaric acid, maleic acid or sodium hydroxide, and not limited to this.

Another aspect of the present invention is to provide a method for upper respiratory tract administration comprising administrating the aforementioned composition to a subject in need at a time point until the composition reaches an effective dose so as to suppress injury upon the subject in need in exposure to toxic gases, wherein the time point is prior to or post exposing the subject in need to the toxic gases.

In the present disclosure, the composition is primarily administrated to an on-site personnel in a fireground or to a personnel in long-term exposure to an environment that may cause cyanide poisoning. In various embodiments, the toxic gases is a mixture of solid particulate matters and gases, wherein the gases contains hazardous substances including free radicals or hydrogen cyanide.

In the present disclosure, the route of administration comprises biological fluid absorption or mucosal absorption. In preferred embodiments, the route of administration comprises nebulizing the composition by a nebulizer so that the subject in need intakes the composition via oral or nasal inhalation. The composition is directly absorbed by cells on the surface of upper respiratory tract, or the composition enters biological fluid on tissue surfaces, such as tissue fluid or mucous, and forms a protective layer before the cells absorbs effective ingredients. In these embodiments, the composition entering the surface biological fluid or remaining on the mucosa surface forms a protective layer. The protective layer neutralizes hazardous substances in the heavy smoke inhaled by the subject in need on the surface. The hazardous substances in blood circulation can be neutralized by the composition that enters blood circulation. In other embodiments, the composition comprises the expectorant, wherein the expectorant eliminates inhaled particulate matters by lowering mucosal viscosity or stimulating upper respiratory epithelial cells to promote mucosal excretion.

In the present disclosure, the subject in need comprises a mammal, wherein the mammal can be exemplified by human, monkey, mouse, rat, rabbit, canine, cat, bovine, horse, porcine, goat, etc.. It should be noted that “effective dose” can be calculated based on the body size, body weight, body surface area, lung volume, alveolar area, bronchial surface area, tracheal surface area and other benchmarks of various mammals. Taking nebulization for example, the effective dose can be calculated based on lung volume of the subject in need. Exemplarily, the averaged lung volume of a mouse is around 1 milliliter. To ensure sufficient amount of cyanide antidote covering the surfaces of alveoli and upper respiratory tract, a C57BL/6 mouse is required to repeat inhalation of the composition at inhaling dose between 25 to 125 mg·L⁻¹ for 1 to 2 minutes. The aforementioned inhaling dose range may variate depending on individual differences. On the other hand, required amount of the cyanide antidote to enter a human adult body is at least 10 to 30 micrograms. In this case, the human adult is necessitated to repeat inhalation of the composition at inhaling dose between 50 to 300 mg·L⁻¹ for 1 to 2 minutes, so that the upper respiratory tract can be protected from damages of hazardous substances such as cyanide or free radicals.

The time point for administration in the present disclosure, taking a personnel in a fireground for example, the personnel inhales the composition to the effective dose 3 to 5 minutes before entering a fireground. The cyanide antidote and the metal ion chelator are anticipated to form a protective layer on the surfaces of tissues, mucosa, alveoli in the upper respiratory tract. The protective layer maintains its protective effect in the next 1 to 2 hours by neutralizing cyanides or blocking free radical production. Thus, acute poisoning or irreversible respiratory tract damage of the personnel in a fireground is minimized. Similarly, a personnel in workplace with risk of cyanide poisoning can also inhale the composition to the effective dose before entering the workplace, or within 1 to 2 hours after leaving from the workplace so as to reverse cyanide poisoning.

In some exemplary embodiments, the subject in need is administrated of the composition for upper respiratory tract administration at dose of 25 to 125 mg·L⁻¹ before exposure to an environment of 50 to 500 ppm cyanide. Survival rate of the subject in need is increased in exposure to cyanide and the protective effect on upper respiratory tract is also achieved. In other exemplary embodiments, the subject in need is administrated of the composition for upper respiratory tract administration at dose of 25 to 125 mg·L⁻¹ before exposure to an environment of 180 to 460 ppm cyanide. In these embodiments, the composition for upper respiratory tract administration is inhaled by the subject in need via nebulization thereof, the inhaling duration is 1 to 2 minutes, and the subject in need is exposed in the cyanide environment for 1 to 45 minutes.

Several examples and experimental examples are listed below to describe the embodiments of the present invention and their technical effects in more detail, but the present invention is not limited by them.

### Test of Mouse Survival Rate

A barrel having volume of 25 liters was prepared, and a tech foam was incinerated inside the barrel so as to produce gaseous cyanides. Then, 50 milliliters gaseous cyanides were withdrawn from the barrel by a syringe, and injected into a vacant bottle having volume of 1250 milliliters for dilution and concentration measuring. The actual cyanide concentration was calculated thereafter.

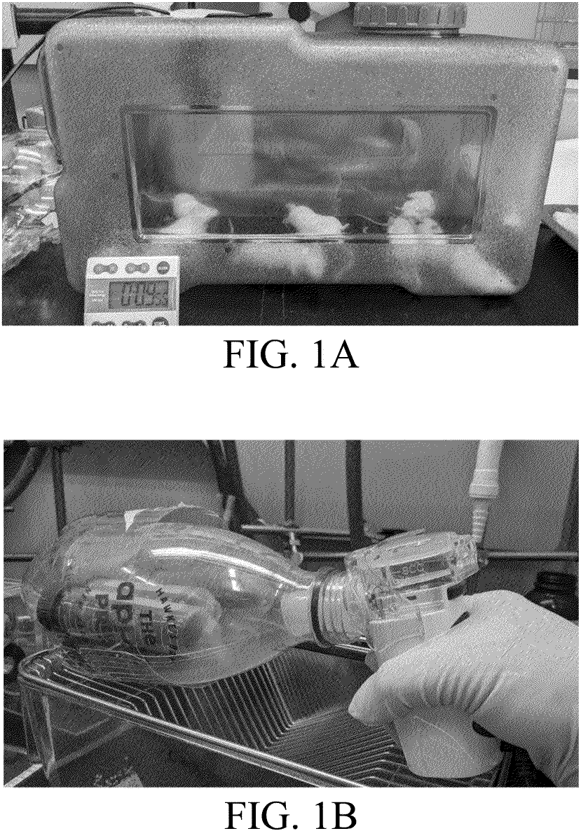

When cyanide concentration in the barrel reached 189.8±5.8 ppm, 30 C57BL/6 mice were placed in the barrel, and 2 liters of 95% oxygen was pumped into the barrel for C57BL/6 mice basic oxygen consumption. FIG. 1A illustrates an example of cyanide exposure barrel in subsequent experimental examples. Please refer to TABLE 1, TABLE 1 illustrates test condition and preliminary test results of mice survival rate test, revealing the lethal dose of cyanide for C57BL/6 mice. On a condition of cyanide concentration at 189.8±5.8 ppm, C57BL/6 mice were removed from the barrel at 20% death rate after inhaling cyanides for 30, 45, and 60 minutes. The corresponding survival rates were 62.5%, 37.5% and 0.0%, respectively.

TABLE 1

| Strain | gender | age (week) | Body weight (g) | Cyanide con. (ppm) | Exposure time (min) | Total number | Survival number | Survival rate (% ) |
|---|---|---|---|---|---|---|---|---|
| C57BL/6 | Male | 8 to 9 | 21.7 ±0.2 | 189.8 ±5.8 | 60 | 10 | 0 | 0.0 |
| | | | | | 45 | 16 | 6 | 37.5 |
| | | | | | 30 | 15 | 7 | 62.5 |
| ICR | male | 8 to 9 | 30.6 ±0.2 | 302.8 ±10.7 | 10 | 6 | 0 | 0.0 |
| | | | | | 7.5 | 8 | 3 | 37.5 |
| | | | | | 5 | 16 | 10 | 46.7 |

On the other hand, survival rate of ICR mice was also assessed. When cyanide concentration in the barrel reached 302.8±10.7 ppm, 41 ICR mice were placed into the barrel, and 2 liters of 95% oxygen was pumped into the barrel for ICR mice basic oxygen consumption. Please continue to refer to TABLE1, the lower columns illustrate test conditions and preliminary test results of mice survival rate test, revealing the lethal dose of cyanide for ICR mice. On a condition of cyanide concentration at 302.8±10.7 ppm, ICR mice were removed from the barrel at 20% death rate after inhaling cyanides for 5, 7.5, and 10 minutes. The corresponding survival rates were 46.7%, 37.5% and 0.0%, respectively.

### Example 1

A composition for upper respiratory tract administration was prepared by mixing 1 ml distilled water, 25 mg hydroxocobalamin and 1 mg deferoxamine.

### Example 2

A composition for upper respiratory tract administration was prepared by mixing 1 ml distilled water, 125 mg hydroxocobalamin and 5 mg deferoxamine.

### Comparative 1

Hydroxocobalamin water solution was prepared by mixing 1 ml distilled water and 25 mg hydroxocobalamin.

### Comparative 2

Deferoxamine water solution was prepared by mixing 1 ml distilled water and 5 mg deferoxamine.

### Experimental Example 1

In the experimental example 1, survival rates of control and experimental groups of C57BL/6 mice were compared. The mice were exposed in an environment containing 184 to 189.8 ppm cyanide for 24 to 39 minutes. The increase of mice survival rates corresponding to each treatment of distilled water, example 1, example 2, comparative 1, and comparative 2 were calculated, respectively.

Before exposure to cyanides, the mice were placed in a 1-liter barrel, and 1 ml distilled water, example 1, example 2, comparative 1, and comparative 2 were respectively nebulized for mice of each group to inhale. Please refer to FIG. 1B, which illustrates an example of mice inhaling the nebulized dose. The control group inhaled the nebulized distilled water, while the experimental groups inhaled the nebulized example 1, example 2, comparative 1 and comparative 2, respectively. After inhalation for 1 to 2 minutes, the mice were exposed in the cyanide-containing barrel for 45 minutes. The mice were removed from the barrel when death rate reached 20%, and were observed for next 24 hours. Results are recorded in TABLE 2.

As shown in TABLE 2, survival rates of C57BL/6 mice who inhaled example 1 or example 2 were significantly increased when compared with that of control group. Survival rates were increased by 22.0% and 80.0%, respectively, while survival rates of mice inhaling comparative 1 or comparative 2 were insignificantly increased.

TABLE 2

| | Gender | age (week) | Body weight (g) | Cyanide conc. (ppm) | Exposure time (min) | Increase of survival rate (%) | |
|---|---|---|---|---|---|---|---|
| Example 1 | Male | 8 | 21.8 ±0.2 | 185.2 ±5.5 | 24.2 ±7.2 | Control | 0.00 |
| | | | | | | experimental | 22.0 |
| Example 2 | Male | 8 | 20.6 ±0.2 | 184 | 34 | Control | 0.00 |
| | | | | | | experimental | 80.0 |
| Comparative 1 | Male | 8 | 20.8 ±0.3 | 184 | 39 | Control | 0.00 |
| | | | | | | experimental | 0.00 |
| Comparative 2 | Male | 8 | 21.5 ±0.4 | 189.8 ±5.8 | 30.5 ±14.5 | Control | 0.00 |
| | | | | | | experimental | 0.00 |

### Experimental Example 2

In the experimental example 2, survival rates of control and experimental groups of ICR mice were compared. The mice were exposed in an environment containing 273 to 320 ppm cyanide for 4.5 to 6.0 minutes. Increase of mice survival rates corresponding to each treatment of distilled water, example 1, example 2, comparative 1, and comparative 2 were calculated, respectively.

Before exposure to cyanides, mice of the control group inhaled the nebulized distilled water, while mice of the experimental groups inhaled the nebulized example 1, comparative 1 and comparative 2, respectively. The procedure for inhalation was the same as that in experimental example 1. After inhalation for 1 to 2 minutes, the mice were exposed in cyanide-containing barrel for 5 minutes. The mice were removed from the barrel when death rate reached 20%, and were observed for next 24 hours. Results are recorded in TABLE 3.

As shown in TABLE 3, the survival rate of ICR mice who inhaled example 1 was significantly increased by 50.0% when compared with that of control group. The survival rate of ICR mice who inhaled comparative 1 was only increased by 20.0%, while the survival rate of ICR mice inhaling comparative 2 dropped by 10.0%.

TABLE 3

| | gender | age (week) | Body weight (g) | Cyanide conc. (ppm) | Exposure time (min) | Increase of survival rate (%) | |
|---|---|---|---|---|---|---|---|
| Example 1 | male | 10 to 11 | 37.4 ±2.0 | 273 ±23.7 | 5.3±0.7 | Control | 0.00 |
| | | | | | | experimental | 50.0 |
| Comparative 1 | male | 8 | 30.0 ±0.2 | 320 | 4.5 | Control | 0.00 |
| | | | | | | experimental | 20.0 |
| Comparative 2 | male | 8 | 31.3 ±0.2 | 277.2 ±8.1 | 5 | Control | 0.00 |
| | | | | | | experimental | -10.0 |

### Experimental Example 3

In experimental example 3, the survival rate of ICR mice after intravenous injection of example 1 was assessed after exposure to cyanide-containing environment. Before exposure to cyanides, mice of control group were injected of distilled water only, while mice of experimental group were injected of 0.1 ml composition from example 1. The mice were exposed in cyanide-containing barrel for 4.5 minutes. The mice were removed from the barrel when death rate reached 20%, and were observed for next 24 hours. Results are recorded in TABLE 4. As shown in TABLE 4, the survival rate of ICR mice after intravenous injection of example 1 was only increased by 20.0% when compared with the survival rate of control group, and the increase of survival rate was far lower than the increase of survival rate in experimental example 2.

TABLE 4

ICR mice-Intravenous injection

| | gender | age (week) | Body weight (g) | Cyanide conc. (ppm) | Exposure time (min) | Increase of survival rate (%) | |
|---|---|---|---|---|---|---|---|
| Example 1 | Male | 9 to 10 | 36.5±0.4 | 319.7 | 4.5 | Control | 0.00 |
| | | | | | | experimental | 20.0 |

### Experimental Example 4

In experimental example 4, the survival rate of ICR mice after intramuscular injection of example 1 was assessed in after exposure to cyanide-containing environment. Before exposure, mice of control group were injected of distilled water only, while mice of experimental group were injected of 1 ml 5X diluted example 1. The mice were exposed in cyanide-containing barrel for 6.0 minutes. The mice were removed from the barrel when death rate reached 20%, and were observed for next 24 hours. Results are recorded in TABLE 5. As shown in TABLE 5, the survival rate of ICR mice after intramuscular injection of example 1 was insignificantly increased when compared with the survival rate of control group, and the increase of survival rate was similarly far lower than the increase of survival rate in experimental example 2.

TABLE 5

ICR mice-Intramuscular injection

| | gender | age (week) | Body weight (g) | Cyanide conc. (ppm) | Exposure time (min) | Increase of survival rate (%) | |
|---|---|---|---|---|---|---|---|
| Example 1 | Male | 9 to 10 | 39.0±1.1 | 305.9 | 6.00 | Control | 0.00 |
| | | | | | | experimental | 0.00 |

### Experimental Example 5

In the experimental example 5, survival rates of control and experimental groups of ICR mice were compared. The mice were exposed in an environment containing 460 ppm cyanide for 1.0 to 2.0 minutes. Increase of mice survival rates corresponding to each treatment of distilled water and example 1 were calculated, respectively.

Before exposure to cyanides, mice of the control group inhaled only the nebulized distilled water, while mice of the experimental groups inhaled the nebulized example 1. The procedure of inhalation was the same as that in experimental example 1. After inhalation for 1 to 2 minutes, the mice were exposed in cyanide-containing barrel for 5 minutes. The mice were removed from the barrel when death rate reached 20%, and were observed for next 24 hours. Results are recorded in TABLE 6.

As shown in TABLE 6, both groups of mice did not survive in 24 hours after they were removed from the barrel, no matter the mice inhaled distilled water only or the composition from example 1.

TABLE 6

| | | | | | | | |
|---|---|---|---|---|---|---|---|
| | gender | age (week) | Body weight (g) | Cyanide cone. (ppm) | Exposure time (min) | Increase of survival rate (%) | |
| Example 1 | Male | 8 | 30.0± 0.2 | 460 | 1 to 2 | Control | 0.00 (no survival) |
| | | | | | | experimental | 0.00 (no survival) |

With respect to experimental example 1 to 5, the composition for upper respiratory administration provided in the present invention successfully increased survival rates of mice exposed in a cyanide-containing environment after the composition was nebulized and inhaled by the mice. Compared with administration of cyanide antidote alone, a composition comprising cyanide antidote and metal ion chelator significantly increased survival rates of the mice. On the other hand, administration of metal ion chelator alone was not only unable to increase survival rate of the mice, but even decreased mice survival rate after exposure to cyanide-containing environment. Obviously, administration of metal ion chelator alone posed negative effect on mice survival rates. The experimental examples as mentioned above demonstrates that the composition for upper respiratory tract administration not only achieved at protective and curing effect via the cyanide antidote, but also significantly enhanced mice survival rate after exposure to cyanide via a dosage form of composition comprising the metal ion chelator.

The composition for upper respiratory tract administration provided in the present invention overcomes limitations of solution injection in prior arts. The composition can be nebulized and inhaled by a subject in need. The composition enters blood circulation rapidly, which allows the subject to use the composition in general situations, a fireground or an environment having trace amount of cyanides. Moreover, the composition protects the subject from smoke injury at a relatively low dose. The composition not only neutralizes cyanides so as to prevent the subject from acute hypoxia, but also blocks free radical chain reaction after the free radicals enter the body. Oxidative stress on the human body is decreased thereby, so lung tissues and integrity of the upper respiratory tract are both preserved. The composition forms a protective layer on the surfaces of upper respiratory tract or alveoli, and blocks smoke hazardous substances from entering the human body. In other words, the composition provided in the present invention can be inhaled by the subject to protect upper respiratory tract prior to exposure in a fireground or a potential cyanide poisoning environment. The composition can further rapidly enter the human body to neutralize cyanides and to block free radical chain reaction when the subject is exposed in the aforementioned environments. In addition, the composition promotes mucous excretion so that particulate matters in smoke are eliminated from the body. Both first aid of acute toxication and precaution prior to cyanide exposure are taken into considerations.

What is claimed is:

1. A composition for upper respiratory tract administration comprising a cyanide antidote and a metal chelator, and the composition is used for cure or protection from fire injury.

2. The composition as claimed in claim 1, wherein the cyanide antidotes is selected from a group consisting of hydroxocobalamin, dicobalt edetate , cobinamide, aquohydroxocobinamide, dinitrocobinamide, methemoglobin, sodium nitrite, amyl nitrite, dimethyl aminophenol, sodium thiosulfate and glutathione.

3. The composition as claimed in claim 1, wherein the metal ion chelator is selected from a group consisting of deferoxamine, deferiprone and deferasirox.

4. The composition as claimed in claim 1, further comprising an expectorant selected from a group consisting of KI, iodinated glycerol, glyceryl guaiacolate, guaifenesin, ambroxol, bromhexine, N-acetylcysteine and lysozyme.

5. The composition as claimed in claim 1, further comprising a carrier, wherein the carrier comprising ionized water, secondary water, ultra-pure water or buffer solution, wherein the buffer solution having a solute selected from a group consisting of phosphate, tris(hydroxymethyl)aminopropanesulfonic acid, dihydroxyethylglycine, tris(hydroxymethyl)aminomethane, tris(hydroxymethyl)methylglycine, 4-hydroxyethylpiperazineethanesulfonic acid acid, n-tris(hydroxymethyl)methyl-2-aminoethanesulfonic acid, 3-(n-morpholine)ethanesulfonic acid, piperazine-n,n′-bis(2-ethanesulfonic acid), dimethylarsinic acid, sodium citrate and 2-morpholineethanesulfonic acid.

6. The composition as claimed in claim 1, wherein the composition is further used for protection and cure confronting with potential exposure to an environment of high concentration cyanide.

7. The composition as claimed in claim 2, wherein the metal ion chelator is selected from a group consisting of deferoxamine, deferiprone and deferasirox.

8. The composition as claimed in claim 7, wherein the composition comprises 5 to 10 parts by weight cyanide antidote and 1 to 5 parts by weight metal ion chelator.

9. The composition as claimed in claim 7, further comprising an expectorant selected from a group consisting of KI, iodinated glycerol, glyceryl guaiacolate, guaifenesin, ambroxol, bromhexine, N-acetylcysteine and lysozyme.

10. The composition as claimed in claim 9, wherein the composition comprises 5 to 10 parts by weight cyanide antidote and 1 to 5 parts by weight metal ion chelator 5 to 10 parts by weight the expectorant.

11. A method for upper respiratory tract administration comprising administrating a composition as claimed in claim 1 to a subject in need at a time point until the composition reaches an effective dose so as to suppress injury upon the subject in need in exposure to toxic gases, wherein the time point is prior to or post exposing the subject in need to the toxic gases.

12. The method as claimed in claim 11, wherein the toxic gases comprises particulate matters, free radicals or hydrogen cyanide.

13. The method as claimed in claim 11, wherein the route of administration comprises biological fluid absorption or mucosal absorption.

14. The method as claimed in claim 11, wherein the subject in need comprises a mammal selected from a group consisting of human, monkey, mouse, rat, rabbit, canine, cat, bovine, horse, porcine and goat.

15. The method as claimed in claim 11, wherein the administration is implemented with a dosage form of a spray, a nebulizer, inhaler, or drops, wherein the inhaler is a metered-dose inhaler, a spray inhaler, or a dry powder inhaler.

16. The method as claimed in claim 11, wherein the toxic gases comprises 50 to 500ppm cyanides.

17. The method as claimed in claim 11, wherein the subject in need is human, and wherein the effective dose is reached by administration of the composition at dose of 50 to 300 mg·L⁻¹ for 1 to 2 minutes, and wherein the subject in need is administrated of the composition 3 to 5 minutes before exposure to the toxic gases.

18. The method as claimed in claim 17, wherein the cyanide antidote is selected from a group consisting of hydroxocobalamin, dicobalt edetate , cobinamide, aquohydroxocobinamide, dinitrocobinamide, methemoglobin, sodium nitrite, amyl nitrite, dimethyl aminophenol, sodium thiosulfate and glutathione, and wherein the metal ion chelator is selected from a group consisting of deferoxamine, deferiprone and deferasirox.

19. The method as claimed in claim 18, wherein the composition further comprises an expectorant selected from a group consisting of KI, iodinated glycerol, glyceryl guaiacolate, guaifenesin, ambroxol, bromhexine, N-acetylcysteine and lysozyme.

20. The method as claimed in claim 19, wherein the composition comprises 5 to 10 parts by weight cyanide antidote and 1 to 5 parts by weight metal ion chelator 5 to 10 parts by weight the expectorant.
